(19) United States
(12) Patent Application Publication
(10) Pub. No.: US 2013/0017969 A1
(43) Pub. Date: Jan. 17, 2013

(54) MARKERS AND METHOD FOR THE DIAGNOSIS OF ROSACEA

(71) Applicants: Jérôme Aubert, Grasse (FR); Martin Steinhoff, San Francisco, CA (US); Michel Rivier, Nice (FR); Johannes Voegel, Chateauneuf/Grasse (FR)

(72) Inventors: Jérôme Aubert, Grasse (FR); Martin Steinhoff, San Francisco, CA (US); Michel Rivier, Nice (FR); Johannes Voegel, Chateauneuf/Grasse (FR)

(73) Assignees: UNIVERSITAT MUNSTER, Munster (DE); GALDERMA RESEARCH & DEVELOPMENT, Biot (FR)

(21) Appl. No.: 13/516,834

(22) PCT Filed: Dec. 16, 2010
(86) PCT No.: PCT/EP2010/069896

§ 371 Date: Oct. 4, 2012

Related U.S. Application Data
(60) Provisional application No. 61/282,113, filed on Dec. 17, 2009.

(51) Int. Cl. C40B 30/04; A61B 10/02

(52) U.S. Cl. 506/9; 600/562

(57) ABSTRACT

Markers for rosacea among the chemokines and cytokines and their receptors, selected from interleukin 8 (IL-8), CXCL1, CXCL2, CXCL3 and CXCL5, the CXCR1 receptor and the CXCR2 receptor are described. Also described, is a method for the diagnosis of rosacea.

The present invention relates to the pharmaceutical field, and in particular the field of markers for rosacea, and also a method for the diagnosis of rosacea.

The invention is based in particular on the fact that the inventors have demonstrated, for the first time, an overexpression both of chemokines and cytokines and of their receptors in rosacea, which is a novel characterization of this pathological condition that has never been described up until now.

Rosacea is a common, chronic and progressive inflammatory dermatosis related to vascular relaxation. It mainly affects the central part of the face and is characterized by redness of the face or hot flushes, facial erythema, papules, pustules, telangiectasia and sometimes ocular lesions called ocular rosacea. In serious cases, particularly in men, the soft tissue of the nose may swell and produce a bulbous swelling known as rhinophyma.

Rosacea generally occurs between the ages of 25 and 70, and it is much more common in people with a light complexion. It affects more particularly women, although this condition is generally more serious in men. Rosacea is chronic and persists for years with periods of exacerbation and remission.

Rosacea was originally called “acne rosacea” because its pustules and its inflammatory pustules greatly resemble those of common acne.

The result of this facial vascular abnormality is a permanent oedema of the dermis, which may be accompanied by an increased colonization by the parasite Demodex folliculorum present on the skin of patients.

Thus, many factors may be involved without necessarily inducing this condition. They are, for example, psychological factors, gastrointestinal disorders, environmental factors (exposure to sunlight, temperature, humidity), emotional factors (stress), dietary factors (alcohol, spices), hormonal factors, vascular factors, or even infection with Helicobacter pilori.

According to the National Rosacea Society, rosacea can be classified into four subtypes plus one variant (erythematotelangiectatic, papulopustular, phymatous and ocular rosacea and a variant known as granulomatous rosacea).

The various rosacea subtypes are taken up below.

First Subtype—Erythematotelangiectatic Rosacea:

It is mainly characterized by episodic erythema and persistent central facial erythema. The appearance of telangiectasia is customary but not essential for a diagnosis of this first subtype. Central facial oedema, burning sensations and squamae are also symptoms that have been reported. Conventionally, patients experience erythrosis attacks due to the abrupt dilation of the arterioles of the face, which then takes on a congestive, red appearance. These attacks can in particular be brought on by emotions, meals and changes in temperature.

Second Subtype—Papulopustular Rosacea:

It is characterized by a persistent central facial erythema with the appearance of central facial papules or pustules. However, the papules and the pustules can also occur in the periorificial regions, i.e. in the perioral, perinasal, or periocular regions. This second subtype resembles common acne, except for the fact that the comedones are absent. Burning sensations may also appear. This subtype has often been seen after or in combination with the first subtype. Telangiectasias are often observed after or with the first rosacea subtype. These telangiectasias may be obscured by the erythema, the papules, or the persistent pustules. Some patients also exhibit oedema on the cheeks and the forehead.

Third Subtype—Phymatous Rosacea

This subtype is characterized by a thickening of the skin and irregular surface nodularities. Rhinophyma most commonly appears, but phymatous rosacea can also appear in other areas such as the chin, the forehead, the cheeks and the ears. Patients suffering from this subtype may also exhibit enlarged and prominent opening of the follicles. This subtype is also often observed after or in combination with subtype 1 or 2, including erythema, telangiectasias, papules and persistent pustules. In the case of rhinophyma, these additional stigmata may be particularly pronounced in the nasal region.

Fourth Subtype—Ocular Rosacea

The diagnosis of rosacea should be considered when the eyes of a patient show one or more of the following signs and symptoms: bloodshot appearance of the conjunctiva, excessive watering, feeling of a foreign body in the eye, burning, dryness, pruritus, photophobia, blurred vision, conjunctival telangiectasias or eyelid margin telangiectasias, periocular erythema, blepharitis, conjunctivitis, and Meibomius gland dysfunction. These signs or symptoms occur before, during or after the appearance of the cutaneous signs. Ocular rosacea is most commonly diagnosed when other cutaneous symptoms are present. However, the cutaneous signs are not necessary for the diagnosis, and studies suggest that the ocular signs and symptoms can occur, in 20% of cases, before the cutaneous manifestations.

Granulomatous Variant:

There is also a granulomatous variant of rosacea which is characterized by hardened yellow, brown or red papules or nodules, and also monomorphic lesions at the site of the papules. Other signs of rosacea may also be present.

Of course, the pathological manifestations of rosacea vary according to the subtype of the disease. However, it will be noted that patients may have characteristics of several different subtypes at the same time. It will also be noted that the disease does not necessarily progress from one subtype to the other (Wilkin et al., 2002, J. AM. Acad. Dermatol. Vol. 46, pages 584-587).

Conventionally, rosacea is treated orally or topically with antibiotics such as tetracyclines, erythromycin, or clindamycin, but also with salicylic acid, antifungal agents, steroids, or metronidazole or with isotretinoin in the severe forms; or with anti-infectives such as azelaic acid.

Thus, the present invention relates to rosacea markers among chemokines and cytokines chosen from interleukin 8 (IL-8), CXCL1, CXCL2, CXCL3, and CXCL5, the CXCR1 receptor and the CXCR2 receptor, and also to a method for the diagnosis of rosacea.

Thus, a first subject of the invention relates to the use of the DNA or the mRNA encoding the chemokines and cytokines chosen from interleukin 8 (IL-8), CXCL1, CXCL2, CXCL3 and CXCL5, the CXCR1 receptor and the CXCR2 receptor, and also the corresponding proteins, so that they can be detected and/or assayed and thus be used as rosacea markers.

IL-8 (CXCL-8) is a member of the family of CXC chemokines, which plays an essential role in the recruitment of neutrophils and other inflammatory cells to the site of inflammation (for a review, see Busch-Petersen J.; Curr Top Med Chem. 2006; 6(13):1345-52). IL-8 has also been described as playing a role in i) in the activation of endothelial cells (induction of proliferation and increase in expression of adhesive molecules: Transactivation of Vascular Endothelial Growth Factor Receptor-2 by Interleukin-8 (IL-8/CXCL8) Is Required for IL-8/CXCL8-induced Endothelial Permeability. D Melissa L. et al (2007) Molecular Biology of the Cell Vol. 18, 5014-5023); ii) in increasing vascular permeability (Transactivation of Vascular Endothelial Growth Factor Receptor-2 by Interleukin-8 (IL-8/CXCL8) Is Required for IL-8/CXCL8-induced Endothelial Permeability. D Melissa L. et al (2007) Molecular Biology of the Cell Vol. 18, 5014-5023) and iii) in neovascularization (IL-8 Directly Enhanced Endothelial Cell Survival, Proliferation, and Matrix Metalloproteinases Production and Regulated Angiogenesis. Aihua Li et al, The Journal of Immunology, 2003, 170: 3369-3376; Autocrine role of IL8 in induction of EC proliferation survival, migration and MMP2 production and angiogenesis Aihua Li et al, Angiogenesis, 2005, 8:63-71; The CXC Chemokine Receptor 2, CXCR2, Is the Putative Receptor for ELR1 CXC Chemokine-Induced Angiogenic Activity. Christina L. Addison et al, The Journal of Immunology, 2000, 165: 5269-5277).

Two chemokine receptors, of the 7-transmembrane domain G protein-coupled receptor family (CXCR1 and CXCR2), are known to be specifically activated by IL-8. Although CXCR2 binds with strong affinity to IL-8 and to the related chemokines such as CXCL6, CXCL1, CXCL2, CXCL3 and CXCL5, CXCR1 binds only to IL-8.

The examples of the present application show an increase, by at least a factor of 2, in the expression of the cytokines (IL8, CXCL2, CXCL3, CXCL5) which target the 2 receptors CXCR2 and CXCR1, thereby demonstrating overall an overexpression both of the chemokines and cytokines and the receptors mentioned and thus representing biological markers characteristic of rosacea.

For the purpose of the present invention, the term “marker” or “biological marker” denotes a biological marker associated with the presence or with the absence of a particular pathological state. The biological markers are in particular proteins, mRNAs or DNAs.

Those skilled in the art are familiar with the methods for analysing and/or detecting and in particular the techniques for quantitatively or semi-quantitatively detecting the mRNA of a gene of interest.

The term “method for analysing and/or detecting” is intended to mean any method which makes it possible to measure the level of gene expression. These methods are generally well known to those skilled in the art and are chosen according to the transcription or translation rates.

The term “transcription rate” is intended to mean the levels of mRNA. The term “translation rate” is intended to mean the levels of protein expression.

The products of expression of the genes/markers (for example proteins) can be analysed by any suitable method, such as western blotting, IHC, mass spectrometry (Maldi-TOF and LC/MS analyses), radioimmunoassay (RIA), Elisa or any other methods known to those skilled in the art or else by assaying the mRNA according to the methods customarily known to those skilled in the art. The techniques based on the hybridization of mRNA with specific nucleotide probes are the most customary (Northern blotting, RT-PCR (Reverse Transcriptase Polymerase Chain Reaction), quantitative RT-PCR (qRT-PCR), RNase protection).

Thus, another aspect of the invention relates to a method for the diagnosis of rosacea, comprising the following steps:

a) taking a biological sample from an individual as described hereinafter,

b) analysing the level of expression of a chemokine or cytokine chosen from IL-8, CXCL2, CXCL3 and CXCL5, of the CXCR1 receptor, of the CXCR2 receptor, in which an overexpression of at least one of these factors is an indicator of rosacea, and thus diagnosing rosacea.

In one alternative embodiment of the invention, the method for the diagnosis of rosacea comprises the following steps:

a) detecting the level of expression of a marker chosen from IL-8, CXCL2, CXCL3, CXCL5, the CXCR1 receptor and the CXCR2 receptor in a sample taken from an individual as described hereinafter,

b) detecting the level of expression of at least one marker chosen from IL-8, CXCL2, CXCL3, CXCL5, the CXCR1 receptor and the CXCR2 receptor in a sample, preferably an equivalent sample, taken from a healthy individual,

c) comparing the difference in level of expression of at least one marker and for which the level of expression is significantly higher than the level of expression in the healthy individual;

d) the overexpression of at least one of the markers being an indicator of rosacea and thus making it possible to diagnose rosacea.

The expression “overexpression of one of the factors or markers” is intended to mean a level of expression increased by at least 50%, and preferably by at least 100%, and even more preferably by at least 200%, or expressed differently, but with equivalent significance, by at least a factor of 2, or at least twice as high as the level in a normal individual; which demonstrates overall an overexpression of the chemokines, the cytokines and the receptors mentioned above, thus representing markers characteristic of rosacea.

According to another aspect of the invention, the latter relates to a method for monitoring the progression of rosacea and thus relates to a method for the prognosis of the progression of rosacea. This method comprises the following steps:

a) taking a biological sample from an individual;

b) analysing the level of expression of a marker chosen from IL-8, CXCL2, CXCL3, CXCL5, the CXCR1 receptor and the CXCR2 receptor in a sample taken and in which a variation in the expression of at least one of these markers is then an indicator of the progression of rosacea.

In the context of the invention, the biological sample corresponds to any type of sample taken from an individual, and can be a tissue sample or a fluid sample, such as blood, lymph or interstitial fluid.

According to one particular and preferred embodiment, the sample is a biopsy of varying size (preferably from 1 to 6 mm in diameter), or a skin sample taken by means of tape stripping, such as with D-Squames, according to the method described in Wong R et al., “Analysis of RNA recovery and gene expression in the epidermis using non-invasive tape stripping”; J Dermatol Sci. 2006 November; 44(2):81-92; or in Benson N R, et al., “An analysis of select pathogenic messages in lesional and non-lesional psoriatic skin using non-invasive tape harvesting”. J Invest Dermatol. 2006 October; 126(10): 2234-41; or else in Wong R et al., “Use of RT-PCR and DNA microarrays to characterize RNA recovered by non-invasive tape harvesting of normal and inflamed skin”. J Invest Dermatol. 2004 July; 123(1):159-67. According to the principle of tape stripping, the product used comprises a flexible translucent polymer support and an adhesive. The product is applied repeatedly to the skin of the patient, preferably until loss of adhesion. The sample obtained relates only to the content of the outermost layers of the epidermis. A method for analysing a protein content obtained in particular according to this sampling method is described in Patent Application WO2009/068825 (Galderma R&D) in order to monitor markers specific for a pathological skin condition and to orient the diagnosis.

Since this method is rapid, non-invasive and relatively inexpensive for detecting the presence of, the absence of or the variation in certain proteomic markers, it is particularly preferred.

This method is in particular characterized by mass spectrometry detection in the skin sample obtained on the flexible and adhesive support in order to detect at least one protein of which the presence, the absence or the variation in amount or in concentration compared with a standard value is associated with the presence, with the progression or with the absence of a particular pathological skin condition.

According to another particular embodiment, the sample may be a hair follicle sampled according to the method described in Patent Application WO2009/053493 (Galderma R&D). This method describes in particular the non-invasive sampling of a hair follicle and also a method for analysing the latter in order to identify the expression profile of the genes or markers.

According to another aspect, the invention relates to a method for monitoring the efficacy of a treatment intended for treating rosacea, and which comprises the following steps:

a) administering the desired treatment to the individual identified as having one or more of the symptoms of rosacea,

b) taking a biological sample from the individual as described above,

c) analysing the level of expression of a marker chosen from IL-8, CXCL2, CXCL3, CXCL5, the CXCR1 receptor and the CXCR2 receptor in the sample taken in b), according to any suitable technique known to those skilled in the art, in which a variation in the expression of at least one of the markers is an indicator in the treatment of rosacea. Preferably, the expression of at least one of the abovementioned markers decreases or moves closer to the level of expression known for a normal individual.

The examples which follow illustrate the invention without limiting the scope thereof.

### EXAMPLE

### Expression and Modulation of IL-8 and of the Related Chemokines and of their Receptors in the Skin of Patients Suffering from Stage I (Erythematotelangiectatic), Stage II (Papulopustular) and Stage III (Phymatous) Rosacea Compared with Volunteers in Good Health

The objective of this example is to measure the amount of chemokine mRNA and in particular IL-8 mRNA in patients suffering from rosacea (stages I to III) and to compare these expression data with those from healthy individuals.

The skin of patients in good health was obtained after plastic surgery (n=6; face). Biopsies of 4 mm were carried out on patients suffering from rosacea presenting stage I (n=10), II (n=10) and III (n=5) (the clinical description of each stage was carried out according to the classification of Wilkin et al., 2002, J. AM. Acad. Dermatol. Vol. 46, pages 584-587) using the biopunch technique, in accordance with good clinical practice. The messenger RNA derived from the various samples was prepared using the RNeasy protect Microkit™ from Qiagen™, according to the manufacturer's method.

The mRNA quality was evaluated using the Agilent RNA 6000 NanoKit according to the manufacturer's instructions.

The expression of the chemokine mRNA and the mRNA of the CXCR1 and CXCR2 receptors was evaluated using the semi-quantitative PCR technology (qRT-PCR—Taqman Low Density Arrays). PCR analyses were carried out using the Cycler 7900 HT machine (Applied Biosystem). The PCR conditions were the following: 40 cycles, 7900 emulation.

The Ct corresponds to the number of PCR cycles which makes it possible to achieve the same level of fluorescence for all the samples. The level of expression is represented in each group by the mean of the Cts and the standard deviation, obtained over all of the samples per group (arithmetic mean +/−standard deviation).

The differential expression between the subtypes compared with the “healthy volunteer” group is measured by means of a mean induction factor (I.F) after standardization of the Cts by means of the expression of three housekeeping genes (glyceraldehyde-3-phosphate dehydrogenase (GAPDH), beta-actin (ACTB) and hypoxanthine phosphoribosyl-transferase 1 (HPRT1)).

For interpreting the results, the following rule applies: the mean number of Cts is inversely correlated with the abundance of mRNA for each gene.

A mean Ct having a low value reflects a strongly expressed gene. Conversely, a mean Ct having a high value indicates that the gene is weakly expressed. Above a value of 35 for the mean Ct, it is considered that the mRNA corresponding to the gene studied is absent from the sample. A mean Ct of between 35 and 30 indicates a weak but detectable expression, a mean Ct of 25 to 30 indicates a moderate expression and, finally, a mean Ct of less than 25 indicates a strong expression of the mRNA corresponding to the gene studied.

TABLE 1
qRT-PCR measurement of the expression of IL-8 and of the related chemokines and of their
receptors in the skin of patients suffering from stage I (erythematotelangiectatic),
stage II (papulopustular) and stage III (phymatous) rosacea compared with volunteers
in good health via the use of the Microfluidic Card technology (Applied Biosystems).

| | Healthy volunteers | | Rosacea subtype I | | Rosacea subtype II | | Rosacea subtype III | |
|---|---|---|---|---|---|---|---|---|
| | Mean | Standard | Mean | Standard | Mean | Standard | Mean | Standard |
| Gene | Ct | deviation | Ct | deviation | Ct | deviation | Ct | deviation |
| name | (n = 6) | (Ct) | (n = 10) | (Ct) | (n = 10) | (Ct) | (n = 5) | (Ct) |
| CXCL1 | 36.1 | 2.1 | 31.8 | 1.6 | 29.9 | 2.3 | 29.8 | 2.5 |
| IL8 | 36.0 | 3.3 | 31.3 | 2.6 | 29.8 | 3.9 | 29.7 | 3.3 |
| CXCL2 | 35.8 | 2.3 | 32.5 | 0.7 | 31.9 | 1.0 | 32.2 | 0.8 |
| CXCL5 | 35.8 | 2.6 | 35.4 | 1.8 | 33.9 | 1.6 | 34.1 | 4.4 |
| CXCL3 | 37.5 | 1.3 | 34.8 | 1.5 | 33.5 | 1.2 | 33.7 | 1.3 |
| CXCR2 | 30.7 | 0.6 | 29.7 | 0.9 | 29.7 | 0.7 | 28.9 | 0.6 |
| CXCR1 | 34.3 | 0.8 | 33.9 | 1.3 | 33.1 | 1.2 | 33.5 | 1.3 |

TABLE 2
Mean relative induction of expression of the
mRNA compared with the “healthy volunteer” group

| | Healthy | Rosacea | Rosacea | Rosacea |
|---|---|---|---|---|
| Gene name | volunteers | subtype I | subtype II | subtype III |
| CXCL1 | 1 | 27 | 22 | 365 |
| IL8 | 1 | 30 | 33 | 251 |
| CXCL2 | 1 | 10 | 10 | 18 |
| CXCL5 | 1 | 1 | 2 | 14 |
| CXCL3 | 1 | 7 | 8 | 8 |
| CXCR2 | 1 | 2 | 2 | 4 |
| CXCR1 | 1 | 2 | 2 | 1 |

The results shown in Tables 1 and 2 demonstrate that the chemokines are absent in the skin of the healthy volunteers (mean Ct greater than 35 PCR cycles). Conversely, in the various rosacea subtypes, the expression of the chemokines is strongly induced. The CXCR1 and CXCR2 receptor mRNAs are detected, respectively, at a low level of expression and a moderate level of expression. A slight induction can be observed in the rosacea patients.

The results in Table 2 demonstrate the overexpression of the cytokines (IL8, CXCL2, CXCL3, CXCL5) which target the 2 receptors CXCR2 and CXCR1, which demonstrates overall an overexpression both of the cytokines and of the receptors in rosacea.

1. A method of diagnosing rosacea, the method comprising diagnosing rosacea using DNA or mRNA encoding chemokines and cytokines selected from the group consisting of interleukin 8 (IL-8), CXCL1, CXCL2, CXCL3, CXCL5, the CXCR1 receptor, the CXCR2 receptor, and corresponding proteins, as markers for rosacea, and correlating the presence of these markers with a diagnosis of rosacea.

2. A method for the diagnosis of rosacea, the method comprising the following steps:
a) taking a biological sample from an individual, and
b) analyzing an expression level of a chemokine or cytokine selected from the group consisting of IL-8, CXCL2, CXCL3, CXCL5, the CXCR1 receptor, and the CXCR2 receptor, in which an overexpression of at least one of these factors is an indicator of rosacea, and
c) diagnosing rosacea based on the analysis of the expression levels.

3. A method for the diagnosis of rosacea, the method comprising the following steps:
a) detecting an expression level of a marker selected from the group consisting of IL-8, CXCL2, CXCL3, CXCL5, the CXCR1 receptor and the CXCR2 receptor, in a sample taken from an individual subject in need of diagnosis,
b) detecting an expression level of a marker selected from the group consisting of IL-8, CXCL2, CXCL3, CXCL5, the CXCR1 receptor and the CXCR2 receptor in a sample taken from a normal individual, and
c) comparing the difference in the expression levels of at least one marker and identifying the markers for which the level of expression is significantly higher in the individual subject in need of diagnosis when compared with the level of expression in the normal individual; and
d) correlating an overexpression of at least one of the markers with the diagnosis of rosacea.

4. The method as defined in claim 2, wherein the overexpression of a receptor or of a cytokine is an expression at a level which is at least twice as high as the level in a normal individual.

5. A method for monitoring the progression of rosacea, the method comprising the following steps:
a) taking a biological sample from an individual subject in need, and
b) analyzing an expression level of a marker selected from the group consisting of IL-8, CXCL2, CXCL3, CXCL5, the CXCR1 receptor and the CXCR2 receptor in the sample taken; and
c) correlating a variation in the level of expression of at least one of the markers with a progression of rosacea in the individual subject.

6. A method of monitoring the efficacy of a treatment for rosacea, the method comprising the following steps:
a) administering a desired treatment to an individual subject having one or more symptoms of rosacea,
b) taking a biological sample from the individual subject, and
c) analyzing an expression level of a marker selected from the group consisting of IL-8, CXCL2, CXCL3, CXCL5, the CXCR1 receptor and the CXCR2 receptor in the sample taken in b); and
d) correlating a variation in the level of expression of at least one of the markers with a level of efficacy for the treatment of rosacea.

7. The method as defined in claim 2, wherein the biological sample is selected from the group consisting of blood, lymph fluid, interstitial fluid, a biopsy of variable size, a skin sample, and a hair follicle.

8. The method of claim 7, wherein the biopsy of variable size has a diameter from 1 mm to 6 mm.

9. The method of claim 7, wherein the skin sample is taken by means of tape or flexible polymer support and adhesive.
